(19) United States
(12) Patent Application Publication
(10) Pub. No.: US 2017/0246190 A1
(43) Pub. Date: Aug. 31, 2017

(54) METHODS AND FORMULATION FOR IMPROVING ORAL AVAILABILITY OF CPT-11 WHILE REDUCING CPT-11 INDUCED GASTROINTESTINAL TOXICITY IN CANCER THERAPY

(71) Applicant: Johnpro Biotech Inc., Taipei City (TW)

(72) Inventors: Pei-Ru LIAO, Taipei City (TW); Kwan-Hwa CHI, Taipei City (TW)

(73) Assignee: Johnpro Biotech Inc., Taipei City (TW)

(21) Appl. No.: 15/519,559

(22) PCT Filed: Oct. 21, 2015
(86) PCT No.: PCT/CN2015/092401

§ 371 Date: Apr. 17, 2017

Related U.S. Application Data
(60) Provisional application No. 62/066,469, filed on Oct. 21, 2014.

(51) Int. Cl. A61K 31/575; A61K 31/4745; A61K 36/28; A61K 31/353

(52) U.S. Cl. CPC A61K 31/575; A61K 31/353; A61K 31/4745; A61K 36/28

(57) ABSTRACT

Disclosed herein are methods and formulation for enhancing oral availability of CPT-11 during cancer therapy while at the same time, reducing its gastrointestinal (GI) toxicity, thus the methods and formulations as disclosed herein may augment the efficacy of cancer therapy.

### FIELD OF THE INVENTION

The present disclosure relates to methods and formulations for improving oral availability of irinotecan or CPT-11 while reducing its gastrointestinal (GI) toxicity (e.g., bloody diarrhea) during the treatment of cancer.

### BACKGROUND OF THE INVENTION

The oral route is often the most convenient way for drug administration, however, many drugs may not be given orally due to their poor bioavailability. These drugs are therefore in general administered via intravenous or intramuscular injection, which requires intervention of a physician or other health care professional, entailing considerable discomfort to the patient, and even requiring administration in a hospital setting with surgical access in the case of certain IV infusions.

It has been speculated that in some cases, the poor bioavailability of a drug is resulted from extensive metabolism via cytochrome P-450 in both liver and small intestines, or fast efflux via epithelium transporters (e.g., P-glycoproteins (P-gps)). Accordingly, it has been postulated that inhibition of P-gp function might be a useful approach for the development of an oral formulation of a drug having poor bioavailability.

Irinotecan hydrochloride or camptothecin (CPT)-11 is one of the few anti-cancer drugs that has been approved for the treatment of colorectal and other gastrointestinal cancers, small cell and non-small cell lung cancer and other malignancies. However, the use of irinotecan is often limited by its poor oral bioavailability and frequent gastrointestinal (G1) toxicity, particularly severe diarrhea generally occurred more than 24 hrs after the administration of irinotecan. In 9-30% of patients receiving irinotecan treatment, the diarrhea does not respond to the well-known anti-diarrhea drug (i.e., loperamide) and thus may require hospitalization, dose modification, and/or interruption of chemotherapy.

CPT-11 is a prodrug that is hydrolyzed in vivo by carboxylesterase (CES) to give an active metabolite, SN-38, which is subsequently glucuronidated by enzymes of UGT1A family, in particular UGT1A1. After ingestion, SN-38 is first converted to SN-38 glucuronide (SN-38G) by hepatic UGT1A and is then excreted into the bile, when SN-38G reaches the GI tract, then is subject to the action of bacterial beta-glucuronidase (βG), which generates free SN-38 that gives rise to GI toxicity (e.g., diarrhea and/or bloody stool). Recent studies found that specific bacterial βG inhibitors may protect mice from CPT-11 induced late diarrhea, which suggests that enterophatic circulation of SN-38 might be the major mechanism leading to intestinal toxicity of CPT-11. Accordingly, a bacterial βG inhibitor is a potential candidate for treating symptoms of CPT-11 induced GI toxicity during a cancer therapy.

In view of the above, there exists in the related art a need of an agent that inhibits the function of bacterial βG so that the oral bioavailability of irinotecan is improved while its GI toxicity is reduced.

### SUMMARY OF THE INVENTION

This invention is concerned with optimization of oral bioavailability of irinotecan or CPT-11 while alleviating its cytotoxicity toward epithelial cells in GI tract. This invention maximizes irinotecan bioavailability by increasing net absorption of CPT-11 in the intestines by suppressing the function of epithelial efflux transporter (e.g., p-glycoprotein), and minimizes GI toxicity by suppressing the activity of intestinal E. Coli beta-glucuronidase (eβG).

Accordingly, the first aspect of the present disclosure is directed to a method for reducing CPT-11 induced gastrointestinal (GI) toxicity in a subject during a cancer therapy. The method includes the step of, administering 1-50 mg/Kg of silychristin to the subject so as to ameliorate or alleviate symptoms of CPT-11 induced GI toxicity.

According to preferred embodiments of the present disclosure, the silychristin is administered to the subject at about 8 mg/kg.

According to some embodiments of the present disclosure, the silychristin is administered prior to, together with, or after the CPT-11 treatment for at least 5 days; preferably, for at least 15 days; and more preferably, for at least 28 days.

According to embodiments of the present disclosure, the subject has a cancer selected from the group consisting of, breast cancer, brain tumor, melanoma, lung cancer, lymphoma, neuroepithelioma, kidney cancer, prostate cancer, stomach cancer, colon cancer, rectal cancer, pancreatic cancer and uterus cancer. In some examples, the cancer is colon cancer or rectal cancer. In other examples, the cancer is metastatic.

According to embodiments of the present disclosure, the CPT-11 induced GI toxicity is diarrhea or bloody stool.

The second aspect of the present disclosure is to provide a method for improving oral availability of CPT-11 while reducing its gastrointestinal (GI) toxicity in a subject underwent a CPT-11 treatment. The method comprises administering to the subject in sequence, one dose of ursodeoxycholic acid (UDCA) that is about 0.1-10 mg/Kg; and at least one dose of silymarin, in which each dose of silymarin is about 1-50 mg/Kg; wherein each doses of silymarin is administered prior to, concurrently with, or after the CPT-11 treatment.

According to embodiments of the present disclosure, the silymarin comprises silychristin.

According to embodiments of the present disclosure, about 0.1-10 mg/Kg of ursodeoxycholic acid (UDCA) is administered to the subject prior to the CPT-11 treatment. Preferably, about 1-5 mg/Kg UDCA is administered to the subject prior to the CPT-11 treatment. More preferably, about 2 mg/Kg UDCA is administered to the subject prior to the CPT-11 treatment.

According to preferred embodiments of the present disclosure, the UDCA is administered to the subject once, prior to the CPT-11 treatment.

According to some embodiments of the present disclosure, total of 5 doses of silymarin are administered to the subject, in which each doses of silymarin is about 8 mg/Kg.

According to other embodiments of the present disclosure, total of 28 doses of silymarin are administered to the subject, in which each doses of silymarin is about 8 mg/Kg.

According to embodiments of the present disclosure, the CPT-11 treatment comprises administering one or more doses of CPT-11 to the subject after the administration of UDCA, in which each dose of CPT-11 is about 0.5-15 mg/Kg. Preferably, one or more doses of CPT-11 is/are administered, with each dose being about 1-10 mg/Kg. More preferably, one dose of CPT-11 about 3 mg/Kg, is administered along with the administration of silymarin.

According to embodiments of the present disclosure, the subject has a cancer selected from the group consisting of, breast cancer, brain tumor, melanoma, lung cancer, lymphoma, neuroepithelioma, kidney cancer, prostate cancer, stomach cancer, colon cancer, rectal cancer, pancreatic cancer and uterus cancer. In some examples, the cancer is colon cancer or rectal cancer. In other examples, the cancer is metastatic.

According to embodiments of the present disclosure, the GI toxicity is CPT-11 induced diarrhea or bloody stool.

It is therefore a further aspect of the present disclosure to provide an oral dosage formulation for treating a cancer, in which the oral dosage formulation is configured to release the active components therein independently at designated time points. The oral dosage formulation of the present disclosure comprises an effective amount of UDCA, CPT-11 and silymarin, and a pharmaceutically acceptable carrier, wherein the oral dosage formulation is configured to release more than 80% of the UDCA within 60 mins, and more than 80% of the CPT-11 within 12 hrs, and more than 80% of the silymarin within 5 days.

According to preferred embodiments of the present disclosure, the silymarin comprises silychristin.

According to preferred embodiments of the present disclosure, in the oral dosage formulation, the silymarin is in a first sustained-release portion that is embedded in a matrix composed by at least one polymer selected from the group consisting of methylcellulose (MC), ethyl cellulose (EC), hydroxypropyl cellulose (HPC), hydroxypropyl methylcellulose (HPMC), carboxyl methylcellulose (CMC), cellulose acetate, cellulose propionate, cellulose acetate propionate, cellulose acetate butyrate, cellulose acetate phthalate, cellulose triacetate, polymethyl methacrylate, polyethyl methacrylate, polyethylene glycol, polyvinyl alcohol, polyvinyl acetate, polyvinyl alcohol-ethylene glycol, carbomer and a combination thereof.

According to preferred embodiments of the present disclosure, in the oral dosage formulation, the CPT-11 is in a second sustained-released portion that is applied as a thin film and deposited on the outer surface of the first sustained-release portion.

According to preferred embodiments of the present disclosure, in the oral dosage formulation, the UDCA is in an immediate-released portion that is applied as a thin film and deposited on the outer surface of the second sustained-release portion.

According to preferred embodiments of the present disclosure, the dosage formulation is in a form of a tablet or a caplet.

The details of one or more embodiments of the invention are set forth in the accompanying description below. Other features and advantages of the invention will be apparent from the detail descriptions, and from claims

It is to be understood that both the foregoing general description and the following detailed description are by examples, and are intended to provide further explanation of the invention as claimed.

### BRIEF DESCRIPTION OF THE DRAWINGS

FIG. 1A is the blood concentration profiles of CPT-11 in animals respectively treated with CPT-11, and the combination of UDCA, CPT-11, and silymarin, in accordance with one one embodiment of the present disclsoure;

FIG. 1B is the blood concentration profiles of SN-38 in animals respectively treated with CPT-11, and the combination of UDCA, CPT-11, and silymarin, in accordance with one embodiment of the present disclsoure;

FIG. 2 are photographs of colon tissues after H&E staining in accordance with one embodiment of the present disclsoure;

FIG. 3A is a bar graph illustrating the effects of the subcomponents of silymarin on the activity of E. Coli βG (eβG) in accordance with one embodiment of the present disclosure;

FIG. 3B is a bar graph illustrating the effects of the subcomponents of silymarin on the activity of human βG (hβG) in accordance with one embodiment of the present disclosure; and

FIG. 4 illustrates the effects of silymarin, silychristin, and saccharic acid 1,4-lactone on the viability of normal cells HEK-293 in accordance with one embodiment of the present disclosure.

### DETAILED DESCRIPTION OF THE INVENTION

The detailed description provided below in connection with the appended drawings is intended as a description of the present invention and is not intended to represent the only forms in which the present invention may be constructed or utilized

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attaching claims are approximations that can vary as desired. At the very least, each numerical parameter should at least be construed in light of the number of reported significant digits and by applying ordinary rounding techniques.

The term “an effective amount” as used herein refers to an amount effective, at dosages, and for periods of time necessary, to achieve the desired result with respect to the enhanced oral bioavailability of CPT-11 (or irinotecan) in a subject during cancer therapy. The specific effective amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., CPT-11, UDCA, silymarin or silychristin of the present disclosure), such as molar concentration, mass concentration, volume concentration, molality, mole fraction, mass fraction and mixing ratio. Specifically, the term “effective amount” used in connection with the drug or compounds described herein refers to the quantity of the drug or compounds, which is sufficient to either increase oral availability of the drug or to alleviate or ameliorate the symptoms associated with the drug-induced GI toxicity in the subject. Persons having ordinary skills could calculate the human equivalent dose (HED) for the medicament (such as the compounds of the present disclosure) based on the doses determined from animal models set forth in the working examples of the present disclosure. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

“Oral bioavailability” of a drug, (e.g., CPT-11 in the present disclosure), is defined as the total amount of the drug systematically available over time after oral ingestion. In the present disclosure, the oral bioavailability of a drug (e.g., CPT-11) is increased by administering at least one other component(s) (e.g., silymarin, UDCA, or both) that suppresses the function of efflux transporters in the intestines, thereby increasing the plasma level of the drug, which is determined by measuring the total systemic drug concentration and/or its active metabolite (e.g., SN-38) overtime after the administration of the drug, and the at least one component(s) that suppresses the function of efflux transporters in the intestines, as compared with that after administration of the drug only. Systemic drug concentration may be determined by any standard measurement techniques, such as high performance liquid chromatography (HPLC). Systemic drug (e.g., CPT-11 and/or its active metabolite SN-38) concentration refers to drug concentration in a mammal's bodily fluids such as serum, plasma or blood, but does not include digestive fluid.

The term “subject” or “patient” refers to an animal including the human species that is treatable with the formulation and/or methods of the present invention. The term “subject” or “patient” intended to refer to both the male and female gender unless one gender is specifically indicated. Accordingly, the term “subject” or “patient” comprises any mammal, preferably a human, which may benefit from the formulations and/or methods of this disclosure.

The term “sustained-release” as used herein refers to the release of the therapeutic compound occurs over an extended period of time leading to lower peak plasma concentrations and/or is directed to a prolonged Tₘₐₓ as compared to “immediate-release”. The term “Tₘₐₓ” as used herein means the time to reach maximum plasma concentration of the active compound or drug (e.g., irinotecan) achieved by the ingestion of the dosage formulation of this invention. The term “AUC₀₋ₜ” refers to an area under the curve from zero to the last measured time point of a measurable drug concentration.

The present invention is based on the discovery that certain compounds (e.g., UDCA or silymarin) are known to increase drug bioavailability by suppressing the expression and/or function of epithelial efflux transporter (e.g., p-glycoprotein), thus these compounds may be co-administered with a drug having poor bioavailability to augment therapeutic effects of the drug in a live subject.

The present invention is also based on the discovery that certain compounds can inhibit bacterial beta-glucuronidase (βG) activity, thereby reduces enterophatic circulation of the active metabolite of a drug (e.g., the active metabolite of CPT-11), thus these compounds may be co-administered with the drug to reduce drug-related GI toxicity.

Accordingly, the first objective of the present disclosure is to provide a method of reducing CPT-11 induced gastrointestinal (GI) toxicity in a subject during a cancer therapy. The method includes the step of, administering 1-50 mg/Kg of silychristin to the subject, so as to ameliorate or alleviate symptoms of CPT-11 induced GI toxicity.

Silychristin is a sub-component of silymarin, which is a mixture of flanonoligans extracted from blessed milk thistle (Silybum marianum) and includes at least silibinin, isosilibinin, silcristin, and silidianin. Accordingly, in optional embodiments, the method of the present disclosure may include administering to the subject an effective amount of silymarin, to ameliorate or alleviate symptoms of CPT-11 induced GI toxicity.

According to embodiments of the present disclosure, silychristin or silymarin is administered to the subject in a dose ranges from about 1 to 50 mg/Kg, such as about 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/Kg; preferably, about 5 to 35 mg/Kg, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 mg/Kg; most preferably, about 8 mg/kg.

According to some embodiments of the present disclosure, the silychristin is administered prior to, together with, or after the CPT-11 treatment for at least 5 days, such as 5, 6, 7, 8, 9, 10, 11, 12, 13, 14, 15, 16, 17, 18, 19, 20, 21, 22, 23, 24, 25, 26, 27, 28, 29, and 30 days; preferably, for at least 15 days, such as 15, 16, 17, 18, 19, 20, 21, 22, 23, 24, 25, 26, 27, 28, 29, and 30 days; and more preferably, for at least 20 days, such as 20, 21, 22, 23, 24, 25, 26, 27, 28, 29, and 30 days; and most preferably, for at least 28 days.

The second aspect of the present disclosure is to provide a method for improving oral availability of CPT-11 while reducing its gastrointestinal (GI) toxicity in a subject underwent a CPT-11 treatment. The method comprises administering to the subject in sequence, one dose of ursodeoxycholic acid (UDCA) that is about 0.1-10 mg/Kg; and at least one dose of silymarin, in which each dose of silymarin is about 1-50 mg/Kg; wherein each doses of silymarin is administered prior to, concurrently with, or after the CPT-11 treatment.

According to embodiments of the present disclosure, the present method includes administering at least one dose of ursodeoxycholic acid (UDCA) to the subject prior to the CPT-11 treatment. Suitable dosage of UDCA that may be used in the present method is about 0.1-10 mg/Kg, such as 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10 mg/Kg; preferably, about 1-5 mg/Kg, such as 1.0, 1.5, 2.0, 2.5, 3.0, 3.5, 4.0, 4.5, and 5.0 mg/Kg; more preferably, about 2 mg/Kg UDCA is administered to the subject prior to the CPT-11 treatment.

According to preferred embodiments of the present disclosure, one dose of UDCA is administered to the subject, prior to the CPT-11 treatment. Alternatively, two doses of UDCA may be administered, respectively before and after the CPT-11 treatment.

Once UDCA is administered, then at least one dose of silymarin may be administered to the subject, either prior to, concurrently with, or after the CPT-11 treatment. According to preferred embodiments of the present disclosure, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 doses of silymarin, which comprises silychristin, is administered to the subject, subsequent to the administration of UDCA. Each dose of silymarin may be administered about 1 to 24 hrs apart, such as 1, 2, 3, 4, 5, 6, 7, 8, 9, 10, 11,12, 13, 14, 15, 16, 17, 18, 19, 20, 21, 22, 23, 24, 25, 26, 27, 28, 29, 30, 31, 32, 33, 34, 35, and 36 hrs apart; preferably, about 8 hrs apart; more preferably, about 12 hrs apart, most preferably, about 24 hrs apart. In one example, total of 5 doses of silymarin are administered, with the first dose being administered concurrently with CPT-11, then another four doses being administered in four consecutive days, with each does being 24 hrs apart. In another example, total of 28 doses of silymarin are administered, with the first dose being administered concurrently with CPT-11, followed by one dose a day, for 27 days.

Suitable dosage of silymarin for use in the present method is about 1 to 50 mg/Kg, such as about 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/Kg; preferably, about 5 to 35 mg/Kg, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 mg/Kg; most preferably, about 8 mg/kg.

According to embodiments of the present disclosure, the CPT-11 treatment comprises administering one or more doses of CPT-11 to the subject, in which each dose of CPT-11 is about 0.5-15 mg/Kg, such as 0.5, 0.6, 0.7, 0.8, 0.9, 1, 2, 3, 4, 5, 6, 7, 8, 9, 10, 11, 12, 13, 14, and 15 mg/Kg. Preferably, one or more doses of CPT-11 is/are administered, with each dose being about 1-10 mg/Kg, such as 1, 2, 3, 4, 5, 6, 7, 8, 9, and 10 mg/Kg. More preferably, one dose of CPT-11 about 3 mg/Kg, is administered along with the administration of silymarin.

Examples of cancer that is treatable by the present method include, but are not limited to, breast cancer, brain tumor, melanoma, lung cancer, lymphoma, neuroepithelioma, kidney cancer, prostate cancer, stomach cancer, colon cancer, rectal cancer, pancreatic cancer and uterus cancer. In some examples, the cancer is colon cancer or rectal cancer. In other examples, the cancer is metastatic.

It will be appreciated that the dosage of CPT-11, UDCA, silychristin, and/or silymarin of the present invention will vary from patient to patient based on factors such as the disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens of the present method may be adjusted to provide the optimal oral bioavailability of CPT-11, and maximum protection toward GI tract.

In one preferred embodiment, the mean maximum plasma concentration (Cₘₐₓ) and the mean AUC₀₋ₜ of CPT-11 is significantly increased when CPT-11 is administered together with UDCA and silymarin in accordance with the method of the present disclosure, as compared with when CPT-11 is administered alone.

A further advantage of administering CPT-11 together with silychristine or silymarin in accordance with the method of the present disclosure is that, GI toxicity that commonly associated with the use of CPT-11, such as diarrhea and/or bloody stool, is significantly alleviated. According to one specific embodiment of the present disclosure, the linings of colon of the animals receiving CPT-11, and silymarin or silychristine, were less damaged; as compared to those of animals receiving CPT-11 treatment alone.

It is therefore a further aspect of the present disclosure to provide an oral dosage formulation for improving the bioavailability of CPT-11, while reducing the CPT-11 induced GI toxicity, during cancer therapy.

The oral dosage formulation of the present disclosure comprises an effective amount of UDCA, CPT-11 and silymarin, and a pharmaceutically acceptable carrier, wherein the oral dosage formulation is configured to release more than 80% of the UDCA within 60 mins, more than 80% of the CPT-11 within 12 hrs, and more than 80% of the silymarin within 5 days.

The oral dosage formulation of the present disclosure may be constructed to comprise,
a first sustained-released portion comprising a first effective amount of silychristin or silymarin;
a second sustained-released portion comprising a second effective amount of CPT-11, which is constructed as a thin film deposited on the first sustained-released portion; and
an immediate-released portion comprising a third effective amount of UDCA, which is constructed as a thin film deposited on the second sustained-released portion.

According to preferred embodiments, the dissolution rate of the immediate-released portion is fast enough that at least about 80% of UDCA is released within 60 mins; while the dissolution rates of the first and second sustained-released portions are slow enough that at least about 80% of the silymarin and CPT-11 are respectively released within 5 days and 12 hrs.

In one example, the dissolution rate of the immediate-released portion is fast enough that more than 80% of UDCA is released within 60 mins, while the dissolution rate of the sustained released portion is slow enough that at least about 80% of silymarin remains unreleased after 12 hours, more preferably at least about 60% of silymarin remains unreleased after 24 hours; most preferably at least 50% of silymarin remains unreleased after 36 hrs. In general, the silymarin will be at least 80% released within 3 days, and will be at least 90% released within 4 days. Accordingly, to preferred embodiments of the present disclosure, the fast released UDCA in the immediate-released portion may suppress the epithelium efflux and thereby enhance the plasma level of CPT-11 or its active metabolite, and the slowly released silymarin in the sustained-released portion may help prevent epithelium tissues of the subject from being damaged by the enterophatic circulated SN-38, thereby reducing CPT-11 induced GI toxicity.

The oral dosage formulation of this invention may be prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

The immediate-released portion of the present oral dosage formulation is designed to rapidly disintegrate upon contacting a fluid such as water and allow fast leaching out of drugs to the environment continuously over a short period of time, such as several minutes or in an hour. The dissolution rate is fast enough that at least 80% of drugs contained therein are released within the first 60 mins In general, at least 90% of drugs contained therein will be released within 2 hours. The drugs in the immediate-release portion may be in a form of an immediate-release particle, or applied as a thin film deposited over the outer surface of the sustained release portion, or a single layer of a tablet constructed in two or more layers, one of the other layers of which is the sustained-release portion.

The immediate-release particles may be produced by any known method, such as dry or wet granulation method as described above. In one example, UDCA is mixed with disintegrants and/or binders, and adsorbents and then the mixture is subjected to either fluid bed granulation or spray drying to produce particles with desired immediate-release property. Examples of disintegrants include, but are not limited to, cross-linked polyvinyl pyrrolidone or crospovidone, starch derivatives such as carboxy methyl cellulose and cellulose derivatives; calcium alginate; carboxymethylcellulose calcium; carboxymethylcellulose sodium; croscarmellose sodium; docusate sodium; hydroxypropyl cellulose; magnesium aluminum silicate; methylcellulose; polacrilin potassium; sodium alginate; sodium starch glycolate and pregelatinized starch. Examples of adsorbents include, but are not limited to, aluminum hydroxide adjuvant; aluminum oxide; aluminum phosphate adjuvant; attapulgite; bentonite; powdered cellulose; colloidal silicon dioxide; hectorite; kaolin; magnesium aluminum silicate; magnesium carbonate; microcrystalline cellulose; pectin; polycarbophil; and saponite. At least 50% of the immediate-release particles thus prepared have a size that may pass 80 mesh; preferably, 60 mesh; more preferably, 40 mesh; and most preferably, 20 mesh.

The sustained-release portion of the present dosage formulation is consitiuted by at least a first sustained-released portion comprising silymarin, and a second sustained-released portion comprising CPT-11, in which the second sustained-released portion is deposited as a thin film on the first sustained-released portion.

According to some embodiments the present disclosure, the silymarin in the first sustained-release portion is embedded in a matrix composed by at least one polymer selected from the group consisting of methylcellulose (MC), ethyl cellulose (EC), hydroxypropyl cellulose (HPC), hydroxypropyl methylcellulose (HPMC), carboxyl methylcellulose (CMC), cellulose acetate, cellulose propionate, cellulose acetate propionate, cellulose acetate butyrate, cellulose acetate phthalate, cellulose triacetate, polymethyl methacrylate, polyethyl methacrylate, polyethylene glycol, polyvinyl alcohol, polyvinyl acetate, polyvinyl alcohol-ethylene glycol, carbomer and a combination thereof.

Alternatively, the first sustained-release portion may contain sustained-release fine particles or pellets that are produced by any known method such as wet granulation or dry granulation method. In one example, the sustained-release fine particles or pellets are produced by wet granulation, particularly, fluid bed granulation. Wet granulation generally involves the steps of mixing the drug, the matrix polymer as described above, a diluent and a binder solution, drying the moist granules, and screening through a suitable sieve to produce particles with desired sizes. Useful binders include, but are not limited to, acacia, tragacanth, alginic acid, sodium alginate, carbomer, carboxymethylcellulose sodium, carrageenan, cellulose acetate phthalate, ceratonia, copovidone, dextrates, dextrin, dextrose, methylcellulose, ethylcellulose, carboxymethyl cellulose, hydroxypropyl methyl cellulose, hydroxyethyl cellulose; hydroxyethylmethyl cellulose; hydroxypropyl cellulose; hydroxypropyl starch; hypromellose, gelatin, starch, sucrose, lactose, magnesium aluminum silicate, maltodextrin, maltose, microcrystalline cellulose, polyvinyl pyrrolidone, polyacrylamide, povidone and pregelatinized starch. Useful diluents include, but are not limited to, ammonium alginate, calcium carbonate, calcium phosphate dibasic, calcium phosphate tribasic, calcium sulfate, cellulose, cellulose acetate, compressible sugar, dextrates, dextrin, dextrose, erythritol; ethylcellulose, fructose, fumaric acid, glyceryl palmitostearate, lactitol, lactose, mannitol, magnesium carbonate, magnesium oxide, maltodextrin, maltose, microcrystalline cellulose, polydextrose, polymethacrylates, sodium chloride, sorbitol, starch, sucrose, sugar spheres, ARBOCEL A300®; LUDIPRESS®; and SUPER TAB®. In certain examples, the sustained-release portion is prepared by mixing silymarin with a sugar sphere made of microcrystalline cellulose (e.g., CELPHERE® CP708), and at least one other matrix polymer as described above, to form silymarin containing granules or pellets. Then, each of the silymarin containing pellets is coated with a second sustained-release film containing CPT-11, and at least a matrix polymer described above (e.g., EUDRAGIT®), a diluent (e.g., talc) and a stabilizer (e.g., triethyl citrate) to produce the desired sustained-release portion. Optionally, the sustained-release portion may be further coated with a protective coating to delay the release of the active ingredients therein, such as silymarin and CPT-11. The protective coating may comprise at least one of the matrix polymer as described above. In one example, the protective coating comprises hydroxymethyl cellulose and polyethylene glycol. In another example, the protective coating comprises triethyl citrate (TEC) and talc. The sustained-release coating and the protective coating may be applied as a film respectivley deposited over the sustained-release pellets and the sustained-release portion, by any known techniques such as spraying, dipping, or pan-coating.

In some optional embodiments, the sustained-release portion and the immediate-release portion respectively prepared by steps as described above are then combined with glidants and lubricants to form the oral dosage formulation of this disclosure. Suitable glidants include, but are not limited to, calcium phosphate, tribasic; calcium silicate; cellulose, powdered; colloidal silicon dioxide; magnesium silicate; magnesium trisilicate; silicon dioxide; starch and talc. Suitable lubricants include, but are not limited to, calcium stearate; glyceryl behenate; glyceryl palmitostearate; magnesium lauryl sulfate; magnesium stearate; polyethylene glycol; potassium benzoate; sodium lauryl sulfate; sodium stearyl fumarate; stearic acid; talc and zinc stearate.

The oral dosage formulation of this disclosure may be in a form of tablets, caplets, bi-layer tablets, film-coated tablets, pills, capsules or the like. Tablets in accordance with this disclosure can be prepared by any mixing and tabletting techniques that are well known in the pharmaceutical formulation industry. In some examples, the dosage formulation is fabricated by direct compressing the respectively prepared sustained-release portion and the immediate-release portion by punches and dies fitted to a rotary tabletting press, ejection or compression molding or granulation followed by compression.

In one example, the dosage form is a single layer tablet containing therein both the sustained-release and the immediate-release portions. In another example, the dosage form is a film-coated tablet having a first sustained-release portion, a second sustained-released portion deposited as a thin film outside the surface of the first sustained-release portion, and a thin film of the immediate-release portion deposited over the outer surface of the second sustained-release portion. The thin film (such as the immediate-released portion and the second sustained -released portion) may be applied as a coating over the first or second sustained-release portion by any known techniques such as spraying, dipping, or pan-coating, or as an additional layer by tabletting or compressing in the same manner as the sustained-release portion. In some examples, the tablet is a scoring tablet having a score line at the center of the tablet for breaking the tablet into two equal halves when necessary. In other examples, the oral dosage formulation is in a form of capsule containing therein both the sustained-release particles or pellets of silymarin or CPT-11, and the immediate-release particles or pellets of UDCA.

Specific examples of the oral dosage formulation of this disclosure include about 10-1,000 mg of CPT-11, such as 10, 20, 40, 50, 60, 70, 80, 90, 100, 150, 200, 250, 300, 350, 400, 450, 500, 550, 600, 650, 700, 750, 800, 850, 900, 950, or 1,000 mg of CPT-11; and about 1-100 mg of UDCA, such as 1, 2, 3, 4, 5, 6, 7, 8, 9, 10, 15, 20, 25, 30, 35, 40, 45, 50, 55, 60, 65, 70, 75, 80, 85, 90, 95, or 100 mg of UDCA; and about 10-1,000 mg of silymarin, such as 10, 20, 40, 50, 60, 70, 80, 90, 100, 150, 200, 250, 300, 350, 400, 450, 500, 550, 600, 650, 700, 750, 800, 850, 900, 950, or 1,000 mg of silymarin.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the respective testing measurements.

The present invention will now be described more specifically with reference to the following embodiments, which are provided for the purpose of demonstration rather than limitation

### EXAMPLES

Materials and Methods

Cell Culture and Animals

Cell line used in the present disclosure were human colon carcinoma cell line HT29, and human embryonic kidney cell line HEK-293. HT29 cells were cultured and maintained in Dulbecco's modified Eagle media (DMEM) supplemented with 10% heat inactivated fetal bovine serum (FBS), 50 units/mL penicillin G, 50 μg/mL streptomycin (pH 7.4), while HEK-293 cells were cultured in complete SFMII growth medium supplemented with 4mM L-glutamine. Both cell lines were maintained in humidified environment comprising 5% CO₂/95% air at 37° C.

NOD/SCID mice (6 weeks old) were obtained from the National Laboratory Animal Center (Taipei, Taiwan) and were kept in a pathogen-free facility with ad libitum access to water and laboratory chow. All animal experiments were performed in accordance with the guidelines of the Animal Welfare Committee of Shin Kong Wu Ho-Su Memorial Hospital (Taiwan, R.O.C.).

Xenograft Tumor Generation and In Vivo Therapy

To inoculate tumor, HT29 cells (1×10⁶ cells per injection) were injected subcutaneously to lab animals to generate s.c. tumors on day 1, which were allowed to grow to about 5 mm in diameter. Two sets of experiments were conducted.

In the first set of experiment, effects of silymarin on the reduction of CPT-11 induced bloody stool were investigated. The animals were randomly assigned to 3 groups, in which animals in group 1 (N=5) were on normal diet; animals in group 2 (N=6) received oral CPT-11 treatment (50 mg/Kg), and animals in group 3 (N=6) received a combined dosage of CPT-11 (50 mg/Kg) and silychristin (10 mg/Kg) for 14 days. The stools of each animals were monitored for at least 17 days. At the end of the experiments, the animals were sacrificed and colon tissues from each animals were stained and used in histological analysis.

In the second set of experiment, oral availability of CPT-11 was evaluated. The animals were randomly assigned to 3 groups, in which animals in group 1 (N=5) were on normal diet; animals in group 2 (N=5) received oral CPT-11 treatment (40 mg/Kg), and animals in group 3 (N=5) received a dose of dose of UDCA (20 mg/Kg) on day 1, an hour later, one dose of CPT-11 (40 mg/Kg) and one dose of silymarin (100 mg/Kg) were given. Blood samples were respectively taken at 1, 4, 8 hrs after the combined treatment of CPT-11 and on days 4, and 8; and the level of SN-38 (i.e., an active metabolite of CPT-11) was measured by HPLC. The body weight (BW) of each animal was measured daily.

After treatment, the animals were sacrificed, and their tumors, livers, and small and large intestines were respectively harvested, weighted and then subject to microscopy and histological analysis.

Histological Analysis

Animal tissues including livers, small and large intestines, duodenum and colon were harvested. Livers were fixed in liquid nitrogen until use. Respective segments of small and large intestines (1-cm each), duodenum and colon (1-cm each) were dissected. These tissue segments were either frozen or fixed in Omnifix® (Melville, N.Y., USA). After fixation, tissues from individual mice were placed in a tissue cassette and processed for embedding in paraffin blocks. Four-micrometer-thick sections were cut and placed on slides. One set of slides was stained using haematoxylin and eosin (H&E), a second set of slides was stained by the periodic acid-Schiff (PAS) reaction and counterstained with haematoxylin to identify mucin in goblet cells. Slides were coded and evaluated by an observer blinded to the group of origin of each slide.

Bloody Diarrhea Evaluation

After treatment, all mice were subjected to daily observation by naked eyes to determine if bloody diarrhea had occurred or not until the end of experiment.

Beta-glucunidase (βG) activity

The βG activity in solution was determined with the fluorogenic substrate 4-methylumbellifery β-D-glucuronide (MUG), in which the non-fluorescent MUG would give rise to glucuronic acid and a highly fluorescent product, 4-methylumbelliferone (4-MU), at higher pH, after being converted by βG. The reaction can be quenched by the addition of a basic solution of sodium carbonate, and pH may be adjusted to quantitate the fluorescent signals. Briefly, the fluorometer was calibrated to an extend that 10 fluorescence units/nM MU was displayed in the presence of the 50 nM 4MU standard The experiment was conducted at, and the assay was started by adding 10 μl of diluted GUS enzyme mixture to the reaction mixture, which was placed in test tubes in water bath at 37° C., whereas 10 μl of extraction buffer without enzyme was used as the control. After 10 minutes, the assays were quenched at 15 second intervals by adding 1.9 ml of the carbonate stopping buffer. The samples were subject to fluorescence measurement, and the data was converted to nmol MUG hydrolyzed as an indication of GUS enzyme activity.

Cell Viability Assay

Cell were cultured at a density of 1.0×10⁵ cells/well in 96-well round-bottom plates (Falcon, UK) containing 200 μL of culturing medium. Cells were maintained for two days at 37° C. in a humidified, 5% CO₂ atmosphere. The proliferation rate of the cells was determined by MTS assay (CellTiter 96 aqueous one-solution cell proliferation assay; Promega, Wis., USA). Forty microliters of CellTiter 96 aqueous one-solution were added to each well. After incubation for 4hrs, the UV absorbance of the solution was measured at a wavelength of 490 nm. All MTS assays were performed in triplicate.

### EXAMPLES

### Example 1

### Combined Administeration of UDCA and Silymarin Increases the Oral Availability of CPT-11

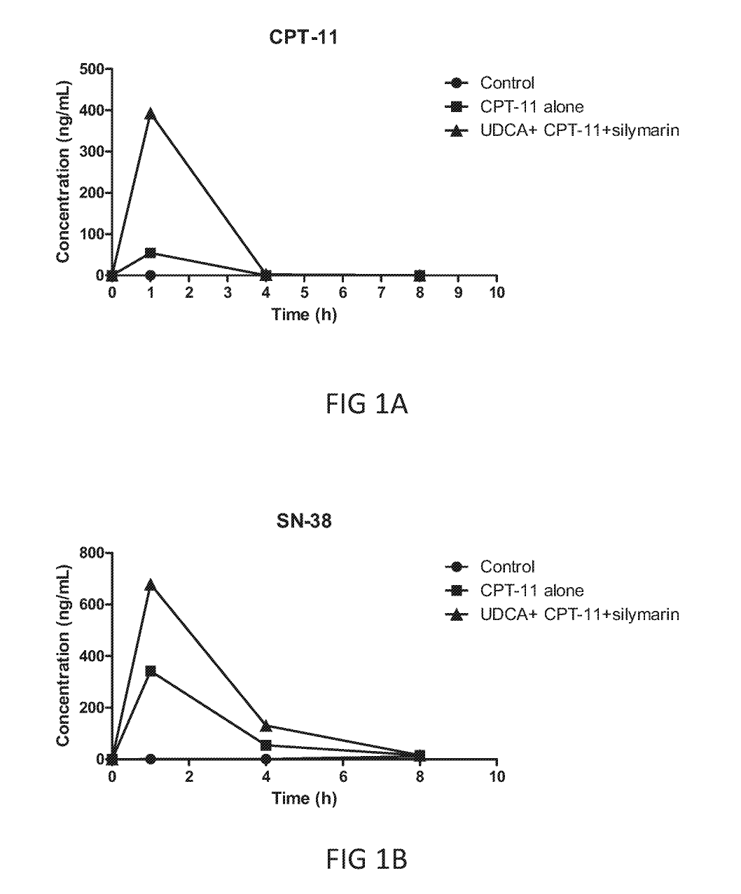

The animals bearing xenografted s.c. colon tumors were randomly assigned into 3 groups, in which group 1 received no treatment, group 2 received one dose of oral treatment of CPT-11 (40 mg/Kg), and group 3 received UDCA (20 mg/Kg), CPT-11 (40 mg/Kg) and silymarin (100 mg/Kg) via oral ingestion in accordance with procedures described in the “Materials and methods” section. Blood samples from each groups were taken at designated time points, and the respective levels of CPT-11 and SN-38 were determined by HPLC. Results are illustrated in FIGS. 1A, and 1B.

After ingestion, CPT-11 is hydrolized by carboxyesterase (CES) to produce an active component, SN-38. As expected, the blood level CPT-11 arised to a higher level 1 hr after ingestion, then quickly faded to an undetectable level in 4 hrs (FIG. 1A). However, a dramatic 8-folds increase in blood CPT-11 level was found (as compared to that of the CPT-11 alone) when the animals received a combo-treatment, in which one dose of UDCA was given prior to the CPT-11 and silymarin treatment (FIG. 1A). This result is a clear indication that the combo treatment of UDCA and silymarin is capable of increasing the oral availability of CPT-11. The results was confirmed by the determination of the blood level of SN-38, which is the active metabolite of CPT-11, in which the combined treatment group exhibited about 2-folds increased in SN-38 (FIG. 1B).

### Example 2

### Silymarin Reduced CPT-11 Induced GI Toxicity

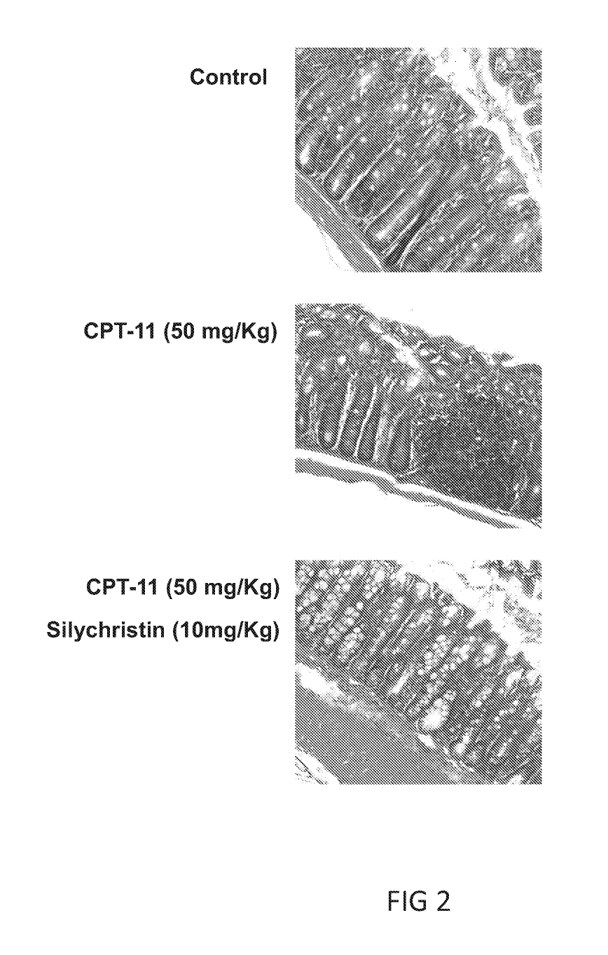

Animals bearing xenografted s.c. colon tumors were randomly assigned into 3 groups, in which group 1 received no treatment, group 2 received one dose of oral treatment of CPT-11 (50 mg/Kg), and group 3 received CPT-11 (50 mg/Kg) and silychristin (10 mg/Kg) via oral ingestion in accordance with procedures described in the “Materials and methods” section Animals were monitored for at least 17 days, then sacrificed and colons tissues from each groups were respectively removed and stained for histological analysis Results are illustrated in FIG. 2.

The histological exmination as depcited in FIG. 2 indicated that the colon tissue sample from CPT-11 treatment animal exhibited severe damage. Specifically, tightly arrayed epithelial cells were found in the control animal, whereas the morphology and integrity of the epithelial cells were disrupted in the CPT-11 treated animal Surprisingly, the damage was rescued by the co-administration of silychristin and CPT-11.

The animals were also subject to daily observation by naked eyes to see if any of the test animals exhibited bloody diarrhea after the treatment. It was found that for animals that received CPT-11 treatment alone, total of 4 animals exhibited bloody stool, respectively on days 12, 14, 17 and 20. Conversely, none of the animals had bloody diarrhea if they were treated with silychristin alone, or the combination of CPT-11 and silychristin. The data is a clear indication that silychristin may effectively alleviate or ameliorate the symptoms of CPT-11 induced GI toxicity.

### Example 3

### Silychristin Inhibits β-glucunidase (βG) Activity

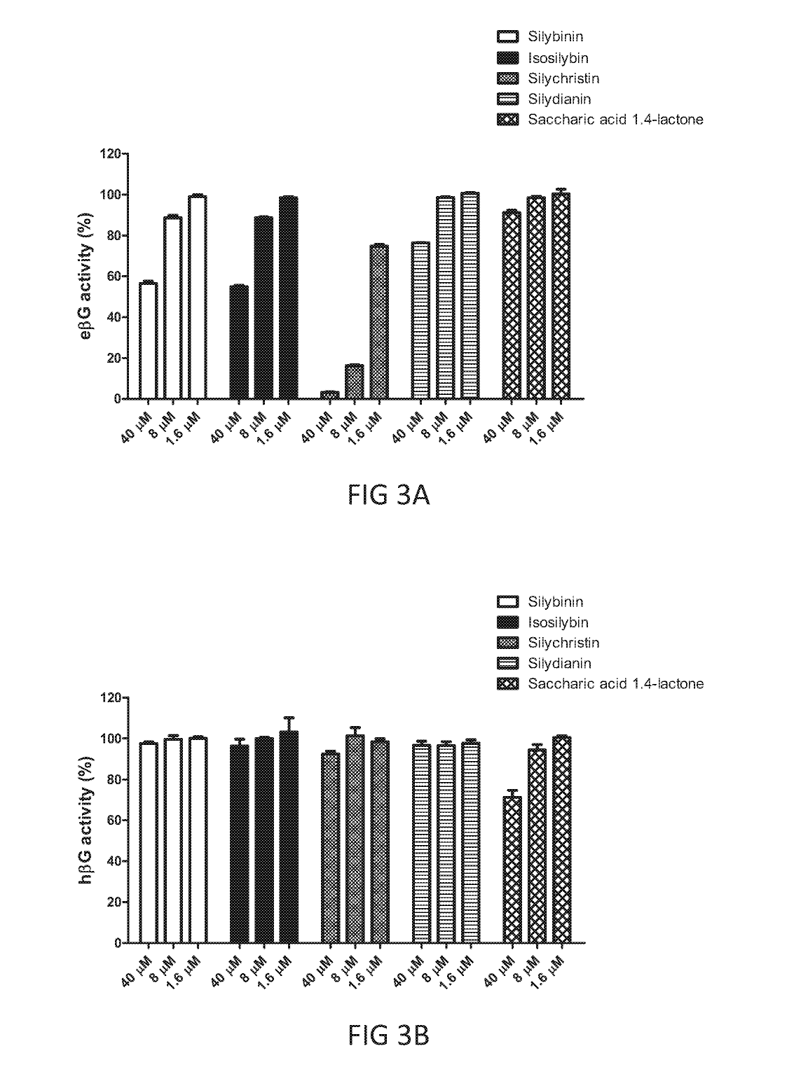

Silymarin is known to be a mixture of flavonolignans extracted from blessed milk thistle (Silybum marianum). The mixture includes at least, silybinin, isosilybinin, silycristin, and silydianin Since results of example 2 demonstrated that silymarin exhibited a protective effect on CPT-i11 induced toxicity, the efficacy of any subcomponent of silymarin toward E. Coli βG (eβG) and/or human βG (hβG) were further investigated in this example in accordance with procedures described in the “Material and Method” section, in which saccharic acid-1,4-lactone, a known βG inhibitor, was included as a positive control. Results are illustrated in FIGS. 3A and 3B.

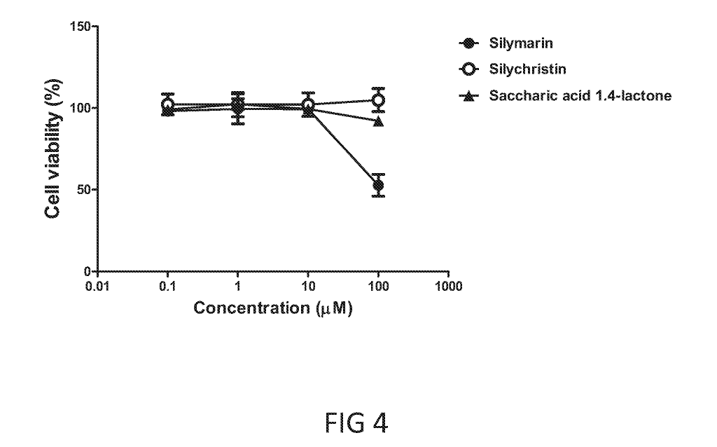

As depicted in FIGS. 3A, among the 4 subcomponents of silymarin, and the three different concentrations that were tested, silychristin at the concentration of 8 μM, was sufficient enough to inhibit nearly 80% of eβG activity without affecting the activity of hβG (FIG. 3B). When the concentration of silychristin was increased to 40 μM, the eβG activity was suppressed to a negligible level. Further, silychristin did not adversely affect normal cells' activities even if the concentration was raised to a relatively high level of 100 μM, at which concentration silymarin exhibited significant adverse effects on cell activities (FIG. 4).

Taken together, the results in FIGS. 4A and 4B confirmed that silychristin is an eβG inhibitor and is responsible for the protective effect of silymarin on CPT-11 induced GI toxicity observed in example 2.

In sum, the data in the present disclosure confirmed that UDCA and silymarin may offer protective effects on the epithelium of colon tissue of an animal subjecting to the treatment of a chemotherapeutic agent, such as CPT-11, and may further alleviate or ameliorate the GI toxicity induced by CPT-11.

The foregoing description of various embodiments of the disclosure has been presented for purpose of illustration and description. It is not intended to be exhaustive or to limit the disclosure to the precise embodiments disclosed. Numerous modifications or variations are possible in light of the above teachings. The embodiments discussed were chosen and described to provide the best illustration of the principles of the disclosure and its practical application to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

1. A method of reducing CPT-11 induced gastrointestinal (GI) toxicity in a subject underwent a CPT-11 treatment, comprising,
administering 1-50 mg/Kg of silychristin to the subject so as to ameliorate or alleviate symptoms of CPT-11 induced GI toxicity.

2. The method of claim 1, wherein the silychristin is administered to the subject at about 8 mg/kg.

3. The method of claim 1, wherein the silychristin is administered prior to, together with, or after the CPT-11 treatment for at least 5 days.

4. The method of claim 1, wherein the silychristin is administered prior to, together with, or after the CPT-11 treatment for at least 28 days.

5. The method of claim 1, wherein the subject has a cancer selected from the group consisting of, breast cancer, brain tumor, melanoma, lung cancer, lymphoma, neuroepithelioma, kidney cancer, prostate cancer, stomach cancer, colon cancer, rectal cancer, pancreatic cancer and uterus cancer.

6. The method of claim 5, wherein the cancer is colon cancer or rectal cancer.

7. The method of claim 5, wherein the cancer is metastatic.

8. The method of claim 1, wherein the subject is a human.

9. The method of claim 1, wherein the CPT-11 induced GI toxicity is diarrhea or bloody stool.

10. A method for improving oral availability of CPT-11 while reducing its gastrointestinal (GI) toxicity in a subject underwent a CPT-11 treatment, comprising administering to the subject in sequence, one dose of ursodeoxycholic acid (UDCA) that is about 0.1-10 mg/Kg; and
at least one dose of silymarin, in which each dose of silymarin is about 1-50 mg/Kg;
wherein each doses of silymarin is administered prior to, concurrently with, or after the CPT-11 treatment.

11. The method of claim 10, wherein the silymarin comprises silychristin.

12. The method of claim 11, wherein the UDCA is administered at about 2 mg/Kg.

13. The method of claim 11, wherein 5 doses of silymarin are administered to the subject, in which each doses of silymarin is about 8 mg/Kg.

14. The method of claim 11, wherein 28 doses of silymarin are administered to the subject, in which each doses of silymarin is about 8 mg/Kg.

15. The method of claim 11, wherein the CPT-11 treatment comprises administering one or more doses of CPT-11 to the subject after the administration of UDCA, in which each dose of CPT-11 is about 0.5-15 mg/Kg.

16. The method of claim 11, wherein the subject has a cancer selected from the group consisting of, breast cancer, brain tumor, melanoma, lung cancer, lymphoma, neuroepithelioma, kidney cancer, prostate cancer, stomach cancer, colon cancer, rectal cancer, pancreatic cancer and uterus cancer.

17. The method of claim 16, wherein the cancer is colon cancer or rectal cancer.

18. The method of claim 16, wherein the cancer is metastatic.

19. The method of claim 11, wherein the subject is a human

20. The method of claim 11, wherein the GI toxicity is CPT-11 induced diarrhea or bloody stool.

21-57. (canceled)
